(19) United States
(12) Patent Application Publication
(10) Pub. No.: US 2007/0270413 A1
(43) Pub. Date: Nov. 22, 2007

(54) Mirtazapine for the treatment of neuropathic pain

(75) Inventors: Andrea Houghton, Newhouse (GB); Gerardus Stephanus Franciscus Ruigt, Oss (NL)

Correspondence Address: ORGANON USA, INC.;PATENT DEPARTMENT, 56 LIVINGSTON AVENUE, ROSELAND, NJ, 07068, US

(73) Assignee: N.V. Organon, Oss (NL)

(21) Appl. No.: 11/804,300

(22) Filed: May 17, 2007

Related U.S. Application Data
(60) Provisional application No. 60/802,430, filed on May 22, 2006.

(51) Int. Cl. A61K 31/55

(52) U.S. Cl. 514/220

(57) ABSTRACT

This invention relates to a method of treatment of neuropathic pain comprising administering to a subject in need of treatment for neuropathic pain a therapeutically effective dose of S(+)-mirtazapine and the use of S(+)-mirtazapine for the manufacture of a medicine for the treatment of neuropathic pain and a pharmaceutical composition for the treatment of neuropathic pain, comprising S(+)-mirtazapine.

### CROSS REFERENCE TO RELATED APPLICATION

This application claims priority under 35 USC 119(e) from U.S. Provisional Application No. 60/802,430, filed on May 22, 2006.

### FIELD OF THE INVENTION

This invention relates to the use of mirtazapine for the treatment of neuropathic pain.

### BACKGROUND OF THE INVENTION

There is both clinical and pre-clinical evidence that anti-depressants are effective for the treatment of painful conditions. In particular, it is used for the treatment of chronic pain (Ansari, Harv Rev Psychiatry, 7 (2000) 257-77; Carter and Sullivan, Curr Opin Investig Drugs, 3 (2002) 454-8; Reisner, Curr Pain Headache Rep, 7 (2003) 24-33; Mattia et al., Minerva Anestesiologica, 68 (2002) 105-114). Especially tricyclic antidepressants (TCAs) are well established in the treatment of difficult conditions such as neuropathic pain and tension type headache (Carter and Sullivan, Curr Opin Investig Drugs, 3 (2002) 454-8; Lynch, J Psychiatry and Neuroscience, 26 (2001) 30-36). However, as TCAs often have severe side effects and lack safety in overdose (Reisner, Curr Pain Headache Rep, 7 (2003) 24-33), a new generation of safer antidepressants with a better tolerability and fewer side effects is increasingly used for treating chronic pain (Ansari, Harv Rev Psychiatry, 7 (2000) 257-77). It has been suggested that 30% of tricyclic antidepressant (TCA) prescriptions are written for the treatment of pain conditions (Su, X, Gebhart, G F (1998) Pain 76: 105-114) and are effective even in the absence of depression (Monks, R Psychotropic Drugs In Textbook of Pain (1994) Eds: PD Wall & R Melzack, Churchill Livingstone, 963-989; McQuay, H. J. and Moore, R. A., Br. Med. J., 314 (1997) 763-764). Indeed, more than 50% of the patients attending the Oxford Pain Clinic are taking antidepressant drugs. However, the mechanisms of the analgesic properties of antidepressants are poorly understood. As the mechanism by which antidepressants exert their antinociceptive effect is still unknown, it is unfortunately difficult to predict which antidepressants will be efficacious in the treatment of pain, and in particular the treatment of neuropathic pain.

Whilst the older tricyclics are known to have actions at a diversity of receptors, the more selective re-uptake blockers e.g. venlafaxine are also effective in the treatment of pain (Sumpton, J. E. and Moulin, D. E., Annals of Pharmacotherapy, 35 (2001b) 557-559). Thus, it is possible that the analgesic effects of these antidepressants are related to their ability to block the re-uptake of serotonin and noradrenaline within the central nervous system (CNS). The effectiveness of duloxetine (Cymbalta™) in treating neuropathic pain (Goldstein et al., Pain 116 (1-2) (2005) 109-118), a dual serotonin and noradrenaline reuptake inhibitor supports this hypothesis.

Mirtazapine is an antidepressive drug, which is marketed for therapy as the racemic mixture, comprising the enantiomers R(−)-mirtazapine and S(+)-mirtazapine. There are some clinical indications which suggest that mirtazapine might be useful in the treatment of pain in humans (Carter and Sullivan, Curr Opin Investig Drugs, 3 (2002) 454-8). Brannon and Stone, J Pain Symptom Manage, 18 (1999) 382-5 report on treatment of a patient with chronic back pain in combination with depression. Bendtsen and Jensen, Neurology, 62 (2004) 1706-11 report on a trial to treat tension headache with mirtazapine; Ansari, Harv Rev Psychiatry, 7 (2000) 257-77 report on treatment of chronic pain with mirtazapine. The use of mirtazapine in the treatment of pain in cancer patients is reported by Theobald et al., J Pain Symptom Manage, 23 (2002) 442-7. In none of these disorders the role of damaged or irritated nerve cells as primary source for the pain is demonstrated. Neuropathic pain is thought to be a consequence of damage to peripheral nerves or to regions of the central nervous system. Wang et al. (US2003/096805) mention mirtazapine in a list of options for topical or local use in combination with local anesthetics to reduce neuropathic pain.

### SUMMARY OF THE INVENTION

The present invention provides for the use of the S(+)-enantiomer of mirtazapine for the treatment of neuropathic pain.

### DETAILED DESCRIPTION OF THE INVENTION

In the context of the present invention, the term “essentially free from R-enantiomer (or R(−)-mirtazapine)” means a content of R(−)-mirtazapine of less than 5%, 2%, 1%, 0.5% or 0.1% of the total amount of mirtazapine.

An embodiment of this invention is the use of pure S(+)-mirtazapine for the manufacture of a medicament of the treatment of neuropathic pain. Pure S(+)-mirtazapine in this context means essentially free from R(+)-mirtazapine.

In the context of the present invention, neuropathic pain means any form of pain associated with a neuropathic disease or condition caused by injury or primary irritation of (part of) a nerve, including degenerative, toxic, metabolic, ischaemic and mechanical forms of injury. Neuropathic conditions include all forms of neuritis and polyneuritis. The neuropathic conditions can be hereditary, such as hereditary sensorimotor neuropathy and hereditary sensory and autonomic neuropathy. Neuropathy can be secondary to diabetes, rheumatic disease or viral infections (such as by the herpes virus; post-herpetic neuralgia). Myofacial pain is a form of neuropathic pain.

According to particular embodiments thereof, the present invention relates to:
the use of S(+)-mirtazapine for the manufacture of a medicament for treatment of neuropathic pain;
a pharmaceutical composition for treatment of neuropathic pain, comprising S(+)-mirtazapine;
a method of treatment of neuropathic pain comprising administering to a subject in need of treatment for neuropathic pain a therapeutically effective dose of S(+)-mirtazapine.

In particular, S(+)-mirtazapine can be used for treatment of pain caused by diabetic neuropathy, which can be a polyneuropathy, comprising all forms of peripheral and central neurological conditions relating to chronic diabetic metabolism disease. In this context, the neuropathy is secondary to diabetes, in that diabetes causes damage to blood vessels providing nutrients etc. to nerves, ultimately leading to damage to the nerves themselves. Therefore, the present invention particularly relates to the use of S(+)-mirtazapine for treatment of pain caused by diabetic neuropathy, as well as to a pharmaceutical composition for treatment of said pain, comprising S(+)-mirtazapine. The invention also relates to a method of treatment of pain caused by diabetic neuropathy, comprising administering a therapeutically effective dose of S(+)-mirtazapine to a subject in need thereof.

S(+)-mirtazapine can, for the purpose of the invention, be used as a free base or as one or more of the commonly accepted acid addition salts. Such compounds can be used in pure form or in admixture with pharmaceutical excipients. Preferred forms of mirtazapine are those in salt form which will result in stable pharmaceutical formulations as reported in WO2005/051714, whereof the maleate salt is preferred.

S(+)-mirtazapine can be prepared in several manners, e.g. by purification from the racemic mixture mirtazapine. Mirtazapine may be prepared using the method described in U.S. Pat. No. 4,062,848. S(+)-mirtazapine can also be obtained by stereoselective synthesis (see WO2005/005410).

In the context of the present invention, the subject is a patient, human or animal, suffering from a condition associated with any one or more of the aforementioned forms of neuropathic pain, to which S(+)-mirtazapine is administered for treating said pain.

The amount of S(+)-mirtazapine, also referred to herein as the active ingredient, which is required to achieve a therapeutic effect, that is the therapeutically effective amount, will vary with the particular compound, the route of administration and the age and other conditions of the subject. The amounts of mirtazapine defined in this description refer to the amount of free base of mirtazapine, unless indicated otherwise.

A suitable daily dose for humans will be in the range of 0.5 to 140 mg, calculated on the weight content of base, per recipient per day, preferably in the range of 5 to 90 mg of the base per recipient per day. In general, parenteral administration requires lower dosages than other methods of administration which are more dependent upon absorption. However, the daily dosages in humans are between 0.01 and 3 mg/kg body weight of the subject.

In the case of tolerance development, treatments can be further optimized by increasing the dose up to 5 times in the course of a chronic treatment in humans. The desired dose may be presented as one, two, three or more sub-doses administered at appropriate intervals throughout the day. A treatment may be for a single day, at discretion of the patient on an “if needed” basis or for a limited determined treatment period defined by a number of days, weeks or months.

While it is possible for the active ingredient to be administered as such, it is preferable to present it as a pharmaceutical formulation. Accordingly, the present invention further provides a pharmaceutical formulation for use in the treatment of neuropathic pain comprising pure S(+)-mirtazapine, together with a pharmaceutically acceptable carrier thereof and optionally other therapeutic agents. The carrier must be “acceptable” in the sense of being compatible with the other ingredients of the formulation and not deleterious to the recipients thereof. The invention further includes a pharmaceutical formulation, as hereinbefore described, in combination with packaging material suitable for the pharmaceutical formulation, said packaging material including instructions for use of the pharmaceutical formulation in the treatment of pain.

Formulations include those suitable for oral or rectal administration. The formulations may be prepared by any methods well known in the art of pharmacy. Such methods include the step of bringing into association the active ingredient with the carrier which constitutes one or more accessory ingredients. Such accessory ingredients include those conventional in the art, such as, fillers, binders, diluents, disintegrants, lubricants, colorants, flavoring agents and wetting agents.

Formulations suitable for oral administration may be presented as discrete units such as tablets or capsules, each containing a predetermined amount of active ingredient; as a powder or granulates; as a solution or suspension. The active ingredient may also be presented as a paste, or may be contained within liposomes or microparticles. The formulation may also be administered as a bolus.

Formulations to be administered parenterally (for example subcutaneously) may also be presented in a suitable sustained release form.

In the context of the present invention, the use of S(+)-mirtazapine for treatment of neuropathy is as stand-alone therapy without any other analgesic co-medication or can be in combination with any other active compound for treatment of the condition to be treated. The other active compound can be administered before, simultaneously with, or after S(+)-mirtazapine. S(+)-mirtazapine can even be combined together with the other active compound into one pharmaceutical composition. The expert will recognise that, in each case of combined use or add-on therapy, the doses and/or formulations may have to be adapted accordingly.

### EXAMPLE

### Effect of S(+)-Mirtazapine in the Chung Neuropathic Model

The Chung Neuropathic Model is an animal model of neuropathic pain. Animals with tight ligation of the L5 spinal nerve develop hyperalgesia and allodynia that can be measured using standard behavioural paradigms.

Chung surgery: animals were anaesthetized with isoflurane administered in oxygen. Under sterile conditions, a longitudinal incision (about 5 mm lateral from the midline) was made at the lower lumbar-sacral level, exposing the paraspinal muscles on the left. The paraspinal muscles were then isolated and removed from the L4 spinous process to the sacrum using small blunt scissors. This opened up the space ventrolateral to the articular processes, dorsal to the L6 transverse process, and medial to the ileum. Small roungers were used to remove the L6 transverse process as completely as possible. The L5 spinal nerve was then isolated and tightly ligated. Upon completion of the operation, haemostasis was confirmed and the wound closed in layers using silk sutures and metal clips. Anaesthesia was then discontinued. Animals were kept in a cage with warm bedding until they had completely recovered from anaesthesia.

The rats' withdrawal threshold to a mechanical stimulus (calibrated von Frey filaments) was measured (baseline reading). Rats were placed on an elevated (˜40 cm) mesh floor in Perspex boxes and filaments of increasing force (2.6-167 mN) were applied to the plantar surface of the paw using an up and down method (Chaplan et al., 1994). The paw was touched with one of a series of 8 von Frey hairs with logarithmically incremental stiffness.

The von Frey hair was presented perpendicular to the plantar surface with sufficient force to cause buckling against the paw, and held for approximately 1-3 seconds. A positive response was noted if the paw was sharply withdrawn. A cut-off of 15 g was selected as the upper limit for testing, since stiffer hairs tend to raise the entire limb rather than buckling, substantially changing the nature of the stimulus.

Following baseline measurements each animal was anaesthetised and the L5 spinal nerve tightly ligated. The animals were allowed to recover from the surgery for a period of at least five days. On the day of drug administration, the paw withdrawal thresholds were re-measured (0 min). Immediately after this reading, the rats were injected subcutaneously with vehicle (1 ml/kg) or S(+)-mirtazapine (0.3-120 μmol/kg) (see Table 1 for doses and group sizes). Readings were then made at regular intervals after compound injection.

Data were expressed as mean±s.e.m. and compared between groups using the Kruskal-Wallis one-way analysis of variance, a non-parametric statistical test. Each of the treatment groups were then compared against the vehicle group, using the non-parametric Dunn's test. The time of maximum effect (Tmax) was calculated.

Subcutaneous administration of S(+)-mirtazapine maleate salt significantly attenuated the mechanical allodynia that develops in this animal model (see Table 1). After administration of the 120 μmol/kg of S(+)-mirtazapine mechanical allodynia was reversed to 53% of the baseline, It was observed that at 120 μmol/kg of S(+)-mirtazapine both vocalisation/struggle on injection (s.c.) and necrosis at site of injection were observed. Further, when rats were returned to holding cages after testing, it was noted that animals in the 60 and 120 μmol/kg group displayed hyperactive/excitable behaviour.

TABLE 1

| | | | | Number | | |
|---|---|---|---|---|---|---|
| | | | | of | | Withdrawal |
| | | Dose | | animals | | threshold (g) |
| Compound | Route | (μmol/kg) | | tested | Tmax | at Tmax |
| 10% Tween 80 | s.c. | 1 | ml/kg | 9 | 60 | 1.2 ± 0.2 |
| S(+)-mirtazapine | s.c. | 0.3 | | 8 | 60 | 1.8 ± 0.4 |
| S(+)-mirtazapine B | s.c. | 3 | | 8 | 60 | 1.7 ± 0.3 |
| S(+)-mirtazapine B | s.c. | 30 | | 8 | 60 | 1.3 ± 0.4 |
| S(+)-mirtazapine B | s.c. | 60 | | 9 | 60 | 5.8 ± 1.0** |
| S(+)-mirtazapine B | s.c. | 120 | | 6 | 60 | 7.4 ± 0.7** |

Table 1 shows the dose groups and number of animals per group, for the neuropathy-induced mechanical allodynia experiments. The table also shows the Tmax for each group and the withdrawal threshold (g) at Tmax. ** denotes p<0.01, when vehicle-treated and compound-treated animals were compared.

### Lack of Effect of R(+)-Mirtazapine in the Chung Neuropathic Model for Comparative Purpose.

Subcutaneous administration of R-mirtazapine (free base) (10-100 μmol/kg) had no effect on the mechanical allodynia induced by ligation of the L5 spinal nerve in rats (see Table 2).

TABLE 2

| | | | | Number of | | Withdrawal |
|---|---|---|---|---|---|---|
| | | Dose | | animals | | threshold (g) at |
| Compound | Route | (μmol/kg) | | tested | Tmax | Tmax |
| 10% Tween 80 | s.c. | 1 | ml/kg | 5 | 30 | 2.3 ± 0.4 |
| R-mirtazapine | s.c. | 10 | | 4 | 30 | 2.3 ± 0.4 |
| R-mirtazapine | s.c. | 30 | | 3 | 30 | 2.1 ± 0.3 |
| R-mirtazapine | s.c. | 100 | | 5 | 30 | 3.2 ± 0.2 |

Table 2: shows the dose groups and number of animals per group, for the neuropathy-induced mechanical allodynia experiments. The table also shows the Tmax for each group and the withdrawal threshold (g) at Tmax. No significant effect was observed.

No side effects were observed after treatment with any of the doses.

### Effect of Rac-Mirtazapine in the Chung Neuropathic Model for Comparative Purpose.

Racemic mirtazapine (10 and 30 μmol/kg) had a small but significant effect on withdrawal threshold (up to 4 g) 40 min after administration when compared to vehicle treated rats (Mann Whitney test, P<0.05). However, this effect was not dose-dependent.

### CONCLUSION

Anti-allodynic activity in rats having mechanical allodynia following L5 spinal nerve ligation was observed with S-mirtazapine following 60 and 120 μmol/kg s.c. In contrast, administration of R-mirtazapine in doses up to 100 μmol/kg s.c. had no effect.

### REFERENCES

Ansari, Harv Rev Psychiatry, 7 (2000) 257-77;
Bendtsen and Jensen, Neurology, 62 (2004) 1706-11
Brannon and Stone, J Pain Symptom Manage, 18 (1999) 382-5
Carter and Sullivan, Curr Opin Investig Drugs, 3 (2002) 454-8;
Goldstein et al., Pain 116 (1-2) (2005) 109-118)
Lynch, J Psychiatry and Neuroscience, 26 (2001) 30-36)
Mattia et al., Minerva Anestesiologica, 68 (2002) 105-114).
McQuay, H. J. and Moore, R. A., Antidepressants and chronic pain-effective analgesia in neuropathic pain and other syndromes, Br. Med. J., 314 (1997) 763-764.
Monks, R Psychotropic Drugs In Textbook of Pain (1994) Eds: PD Wall & R Melzack, Churchill Livingstone, 963-989;
Reisner, Curr Pain Headache Rep, 7 (2003) 24-33
Su X, Gebhart GF (1998) Effects of tricyclic antidepressants on mechanosensitive pelvic nerve afferent fibers innervating the rat colon. Pain 76:105-114.
Sumpton, J. E. and Moulin, D. E., Treatment of neuropathic pain with venlafaxine, Annals of Pharmacotherapy, 35 (2001) 557-559.
Theobald Del. Kirsh KL. Holtsclaw E. Donaghy K. Passik S. D. An open-label, crossover trial of mirtazapine (15 and 30 mg) in cancer patients with pain and other distressing symptoms. Journal of Pain & Symptom Management. Vol. 23(5)(pp 442-447), 2002.
Van der Burg U.S. Pat. No. 4,062,848
Wang et al. (US2003/096805)
WO2005/051714
WO2005/005410

What is claimed:

1. A pharmaceutical composition for treatment of neuropathic pain, comprising S(+)-mirtazapine.

2. A pharmaceutical composition according to claim 2, whereby the pain is caused by diabetic neuropathy.

3. A method of treatment of neuropathic pain comprising administering a therapeutically effective dose of S(+)-mirtazapine to a subject in need of treatment for neuropathic pain.

4. The method according to claim 3, wherein the subject suffers from diabetic neuropathy.
